(19) United States
(12) Patent Application Publication
(10) Pub. No.: US 2019/0117299 A1
(43) Pub. Date: Apr. 25, 2019

(54) ABLATION CATHETER WITH MICROELECTRODE AND METHOD FOR PRODUCING THE SAME

(71) Applicant: VASCOMED GMBH, BINZEN (DE)

(72) Inventor: RALF KAUFMANN, LOERRACH (DE)

(21) Appl. No.: 16/157,697

(22) Filed: Oct. 11, 2018

(30) Foreign Application Priority Data
Oct. 23, 2017 (DE) 102017124651.7
Feb. 14, 2018 (EP) 18156706.6

(51) Int. Cl. A61B 18/14

(52) U.S. Cl. CPC A61B 18/1492; A61B 2017/00526

(57) ABSTRACT

An ablation catheter with a catheter shaft, an ablation electrode that is arranged at the distal end of the catheter shaft, a microelectrode that is arranged on a surface of the ablation electrode, and a lead element that has an electrically conductive connection with the microelectrode. The lead element is surrounded by an insulating material so that the lead element is electrically insulated from the ablation electrode. At least sections of the insulating material with the lead element are arranged on the surface of the ablation electrode. The lead element is fastened by tensioning.

### CROSS-REFERENCE TO RELATED APPLICATION

This application claims the priority, under 35 U.S.C. § 119, of German application DE 10 2017 124 651.7, filed Oct. 23, 2017 and of European application EP 18156706.6, filed Feb. 14, 2018; the prior applications are herewith incorporated by reference in their entireties.

### BACKGROUND OF THE INVENTION

### Field of the Invention

The disclosure relates to an ablation catheter with a microelectrode and a method for producing the same.

Intracardiac mapping (e.g., by measuring the impedance between catheter electrodes) in the contact zone between an ablation electrode of an electrophysiological catheter and the cardiac tissue is known. So-called “micromapping” by use of one or more microelectrodes on the catheter is a promising approach to be used for the purposes of medical diagnosis and monitoring during ablation to treat cardiac arrhythmia.

U.S. patent publication No. 2004/0092806 A1 discloses an ablation catheter with microelectrodes. The metal microelectrodes having the shape of cylinders, cones, or mushrooms are inserted in holes in the ablation electrode, and are electrically insulated from the ablation electrode, and are provided with electric leads inside the ablation catheter.

U.S. patent publication No. 2008/0243214 A1 also discloses an ablation catheter with microelectrodes. The microelectrodes are cylindrically shaped, and are arranged in holes in the catheter head.

Published, European patent application EP 3 015 064 A2, corresponding to U.S. Pat. Nos. 9,314,208, 10,039,494 and 9,693,733, discloses an ablation catheter with indentations to hold microelectrodes.

U.S. patent publication No. 2014/0081111 A1 discloses another ablation catheter with microelectrodes.

Other catheters are described in European patent applications EP 3 040 043 A1 (corresponding to U.S. Pat. No. 10,034,707), EP 3 009 092 A1 (corresponding to U.S. patent publication No. 2016/0100878), published, non-prosecuted German patent application DE 100 08 918 A1 (corresponding to U.S. Pat. No. 6,595,991), and international patent disclosure WO 2017/070559 A1 (corresponding to U.S. patent publication No. 2017/0112405).

The known microelectrode catheter concepts require a relatively large amount of space for the microelectrodes on the ablation electrode, especially for the fastening, the electrical insulation, and for making contact with the electrical leads. In the case of irrigated ablation catheters, the shape and mounting of the electrical lead routes greatly interfere with the routing of the irrigation tubes and the shape of the irrigation channels.

### SUMMARY OF THE INVENTION

An object is to provide improved technologies for ablation catheters. In particular, one aim may be to reduce the space required for a microelectrode and its lead.

An ablation catheter according to the independent device claim and a method for producing an ablation catheter according to the independent method claim are disclosed. Other embodiments are the subject of the dependent claims.

One aspect involves providing an ablation catheter. The ablation catheter comprises a catheter shaft and an ablation electrode arranged at a distal end of the catheter shaft. Furthermore, a microelectrode and a lead element are provided. The microelectrode is arranged on a surface of the ablation electrode. The lead element has an electrically conductive connection with the microelectrode. The lead element is surrounded by an insulating material so that the lead element is electrically insulated from the ablation electrode. At least sections of the insulating material with the lead element are arranged on the surface of the ablation electrode. The lead element is fastened to the ablation electrode by tensioning.

Another aspect of the disclosure relates to a method for producing an ablation catheter. The method comprises the following steps: providing a catheter shaft, arranging an ablation electrode on a distal end of the catheter shaft, arranging a microelectrode on a surface of the ablation electrode, and providing a lead element that has an electrically conductive connection with the microelectrode. The lead element is surrounded by an insulating material, so that the lead element is electrically insulated from the ablation electrode. At least sections of the insulating material with the lead element are arranged on the surface of the ablation electrode. The lead element is fastened to the ablation electrode by tensioning.

Multiple microelectrodes can be arranged on the ablation electrode, and each of the multiple microelectrodes can be electrically insulated from the ablation electrode. Each of the multiple microelectrodes can be connected with a dedicated lead element. It can also be provided that multiple microelectrodes are connected with a common lead element. The lead element of every microelectrode and/or common lead elements can be fastened by tensioning. Mixed forms are also possible. For example, a first group of multiple microelectrodes can be connected with a common first lead element, and a second group of multiple microelectrodes can be connected with a common second lead element. The microelectrodes can have varied shapes, e.g., round, oval, or semicircular. The microelectrodes can point in various directions on the surface of the ablation electrode.

The microelectrode can be arranged on an end face of the ablation electrode or on a lateral surface of the ablation electrode. If multiple microelectrodes are provided, one or more microelectrodes can be arranged on the end face of the ablation electrode. Additionally or alternatively, one or more microelectrodes can be arranged on the lateral surface of the ablation electrode. Multiple microelectrodes can be uniformly distributed on the end face and/or the lateral surface of the ablation electrode. The end face of the ablation electrode can also be referred to as the distal end of the ablation electrode.

The lead element can be tensioned in a recess on the ablation electrode. The lead element can be fastened so that a first section of the lead element is tensioned in a first recess on a first side of the lateral surface, a second section of the lead element is tensioned in a second recess on the end face, and a third section of the lead element is tensioned in a third recess on a second side of the lateral surface. The first side and the second side can lie opposite one another on the lateral surface. The first recess, the second recess, and the third recess can form a continuous recess in which the lead element is tensioned.

Two or more lead elements symmetrically arranged on the electrode periphery are acted on by tensile forces F in the direction from distal to proximal. They are fastened inside the catheter and do not have to meet the high biocompatibility requirements of the outside surface, and are held under tensile stress. The way of fastening the lead element makes it possible to reduce or even completely eliminate the use of an adhesive. In one embodiment, the fastening of the lead element is free of an adhesive.

The distal end of the catheter shaft is understood to be the end that is introduced into the patient's body when the catheter is used (for example, during ablation). The distal end of the catheter shaft with the ablation electrode can also be referred to as the catheter head. The proximal end of the catheter shaft is the end that remains outside the body when the catheter is used. The proximal end of the catheter shaft can have a catheter handle formed on it. The catheter handle can have a connection device on it to connect the ablation electrode and the microelectrode(s) to a control device.

It can be provided that at least sections of the lead element surrounded by the insulating material are arranged in a recess formed on the surface of the ablation electrode, for example that they are tensioned in it. It can also be provided that the microelectrode and the lead element surrounded by the insulating material are arranged in a recess formed on the surface of the ablation electrode. The microelectrode and the insulating material surrounding the microelectrode can be considered to be embedded into the surface of the ablation electrode. The recess can be formed on the end face and/or on the lateral surface of the ablation electrode. If multiple microelectrodes are provided, multiple recesses can be formed on the surface of the ablation electrode, so that every microelectrode and/or the respective lead element are arranged in one of the multiple recesses, and are, for example tensioned in it. The recess (or possibly the recesses) can have a depth of 0.05 mm or less (e.g., 0.03 mm or 0.01 mm).

The microelectrode and/or the lead element surrounded by the insulating material can be glued into the recess. Alternatively, the microelectrode and/or the insulating material can be fastened using a way of fastening involving clamping, tensioning, shrinking, or stretching. Other possible ways of fastening the microelectrode and/or the lead element surrounded by the insulating material are also conceivable.

The microelectrode can be electrically insulated from the ablation electrode, for example by the insulating material.

The insulating material can be polyimide (PI), polyurethane (PUR), polyether block amide (PEBA), or a liquid crystal polymer (LCP). Liquid crystal polymers are simple to process (they are still durable and dimensionally stable for a short time even at 100° C.) and are biocompatible, which makes them especially suitable for use in an ablation catheter. The insulating material can be provided in the form of a flexible film material, e.g., in the form of an LCP film or a film made from one of the other previously mentioned materials.

The microelectrode and/or the lead element surrounded by the insulating material can be arranged in a form-fit (or exact-fit) manner in the recess.

The lead element can contain a metal (e.g., copper) or a metal alloy, or can consist of a metal (e.g., copper) or a metal alloy. The lead element can be in the form of a conductor track. In one embodiment, the lead element is in the form of a copper conductor track, which is surrounded by a flexible LCP film for electrical insulation.

The microelectrode (or the microelectrodes) can contain a metal (e.g., copper) or a metal alloy, or can consist of a metal (e.g., copper) or a metal alloy. The microelectrode can be coated, for example with a metal (e.g., gold, platinum, or another electroplatable biocompatible metal) or a metal alloy. The microelectrode can be in the form of a planar microelectrode, the extension of the microelectrode being substantially larger in two dimensions (the surface area of the microelectrode) than in the third dimension (height of the microelectrode). The extension in the planar direction (e.g., the diameter of the microelectrode) can be 0.3 mm. For example, the microelectrode (or possibly the microelectrodes) can be in the form of a planar (or slightly bulging) microelectrode made of copper that is coated with gold.

The dimensions are oriented on the basis of the circumference and the axial length of the ablation electrode (e.g., circumference U=7 mm and length L=3 to 8 mm). The maximum electrode diameter D of the microelectrodes including insulation edges is D=U/n, where n is the number of microelectrodes. What is important is the proportion of the surface area of the ablation electrode that is covered by the microelectrodes including insulation edges (and thus not effective). This proportion should be less than about a third. The microelectrodes can have a peripheral shape that makes the free contact surface of the ablation electrode large enough for efficient delivery of ablation current to the cardiac tissue in every roll and tilt angle position.

It can be provided that the microelectrode and the lead element surrounded by the insulating material are flush with the surface of the ablation electrode. In particular, a transition from the microelectrode and/or the insulating material to the surface of the ablation electrode can be free of unevenness or edges.

The lead element surrounded by the insulating material can be arranged on the surface of the ablation electrode from the microelectrode all the way to the distal end of the catheter shaft, and be routed in an interior of the catheter shaft at the distal end of the catheter shaft. At the distal end of the catheter shaft there is a transition from the material of the ablation electrode (as a rule a metal, e.g., gold or platinum, or a metal alloy) to the material of the catheter shaft (e.g., a plastic). At this transition, the lead element with the insulating material can be routed into the interior of the catheter shaft.

The ablation electrode can have an irrigation opening formed in it. The irrigation opening can be connected with an irrigation tube arranged in the interior of the catheter shaft. The ablation electrode can also have multiple irrigation openings formed in it, which are connected with the irrigation tube. Arrangements and geometries of irrigation tubes are known. For example, the embodiments disclosed in published, European patent application EP 2 380 517 A1 (especially the variants shown in FIGS. 3B and 4B) can be combined with this disclosure.

The microelectrode can be formed with a hole and be arranged on the surface of the ablation electrode in such a way that at least sections of the hole of the microelectrode are arranged on the irrigation opening. In this case, irrigation can be performed through the hole in the microelectrode. This can have the following advantages:

1. Increased use of surface area and other placement possibilities for microelectrodes.
2. Protection from overheating and the resulting thrombus formation at the material transitions from the insulation edge (polymer) to the micro electrode and ablation electrode.

The microelectrode can be in the form of a ring electrode that surrounds the ablation electrode around its periphery.

The microelectrode can be in the form of a toothed microelectrode. A toothed microelectrode has two sections. A first section has multiple projections, which are spaced apart from one another. A second section also has multiple projections, which are spaced apart from one another. The first section and the second section are arranged opposite one another and offset to one another, so that the projections of the first section point in opposite directions from the projections of the second section, the projections of the first section being arranged in the empty spaces between the projections of the second section, and the projections of the second section being arranged in the empty spaces between the projections of the first section. It can be provided that from outside only a chain of microelectrodes is visible on the periphery of the ablation catheter, without the microelectrodes having a visible connection. However, the microelectrodes are connected to only two electrical poles (connections between the microelectrodes) in alternation.

The width of the microelectrodes, and thus their number and separation on the periphery, can be optimized so that it is possible to measure clear microimpedances or electrical excitation fields from every axial rotation position of the ablation electrode.

The microelectrode can be combined with a thermocouple in the same position. The thermocouple is electrically insulated from the microelectrode, and can be encapsulated into the insulating material (e.g., LCP) under the microelectrode. This allows, for example, simultaneous capture of tissue temperature and microimpedance during the ablation.

Features that are disclosed for the microelectrode can be transferred to embodiments with multiple microelectrodes. The features that are disclosed in connection with the ablation catheter can be applied analogously to the method for producing the ablation catheter, and vice versa.

Other features which are considered as characteristic for the invention are set forth in the appended claims.

Although the invention is illustrated and described herein as embodied in a ablation catheter with a microelectrode and a method for producing the same, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE SEVERAL VIEWS OF THE DRAWING

FIG. 1 is a diagrammatic, perspective view of an ablation catheter with microelectrodes;

FIG. 2 is a perspective view of a catheter head of the catheter in FIG. 1;

FIG. 3 is a cross sectional view of the catheter head in FIG. 2;

FIG. 4 is a schematic representation of a microelectrode;

FIG. 4A is a sectional view taken along the section line IVA-IVA shown in FIG. 4;

FIG. 4B is a sectional view taken along the section line IVB-IVB shown in FIG. 4;

FIG. 5 is a schematic representation sectional view of another embodiment of the microelectrode;

FIG. 5A is a sectional view taken along the section line VA-VA shown in FIG. 5;

FIG. 5B is a schematic representation sectional view of another embodiment of the microelectrode;

FIG. 5C is a sectional view taken along the section line VC-VC shown in FIG. 5B;

FIG. 5D is a schematic representation sectional view of further embodiment of the microelectrode;

FIG. 5E is a sectional view taken along the section line VE-VE shown in FIG. 5D;

FIG. 6 is a schematic representation of an additional embodiment of the microelectrode;

FIG. 6A is a sectional view taken along the section line VIA-VIA shown in FIG. 6;

FIG. 6B is a sectional view taken along the section line VIB-VIB shown in FIG. 6;

FIG. 7 is a perspective view of the catheter head with microelectrodes according to the embodiment according to FIG. 6;

FIG. 8 is a perspective view of the catheter head, the microelectrodes being in the form of ring electrodes;

FIG. 8A is a sectional view taken along the section line VIIIA-VIIIA shown in FIG. 8;

FIG. 8B is a sectional view taken along the section line VIIIB-VIIIB shown in FIG. 8;

FIG. 9 is a perspective view of the catheter head, the microelectrodes being in the form of toothed microelectrodes;

FIG. 9A is a sectional view taken along the section line IXA-IXA shown in FIG. 9;

FIG. 10 is a schematic representation of the connections of the lead elements to the toothed microelectrodes from FIG. 9;

FIG. 11 is a perspective view of a first type of fastening of the microelectrodes to the ablation electrode;

FIGS. 11A is a sectional view of the first fastening of the microelectrodes to the ablation electrode;

FIG. 11B is a sectional view of the first fastening of the microelectrodes to the ablation electrode;

FIG. 12 is a perspective view of a second type of fastening of the microelectrodes to the ablation electrode;

FIG. 13 is a perspective view of a third type of fastening of he microelectrodes to the ablation electrode;

FIG. 14 is a schematic representation of an embodiment of the microelectrode with a thermocouple; and

FIG. 14A is a section view taken along the section line XIVA-XIVA shown in FIG. 14.

### DETAILED DESCRIPTION OF THE INVENTION

The same reference numbers are used for the same components.

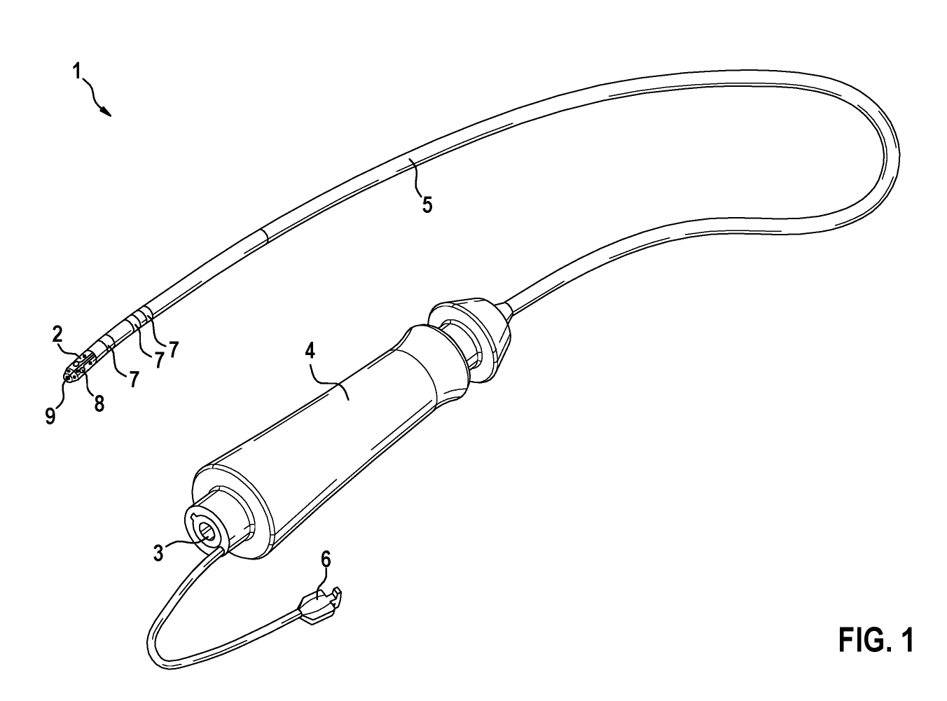

Referring now to the figures of the drawings in detail and first, particularly to FIGS. 1-3 thereof, there is shown a sample embodiment of an ablation catheter 1 with a microimpedance measurement function, for example in order to carry out micromapping for diagnostic purposes and/or for monitoring of lesion formation during ablation.

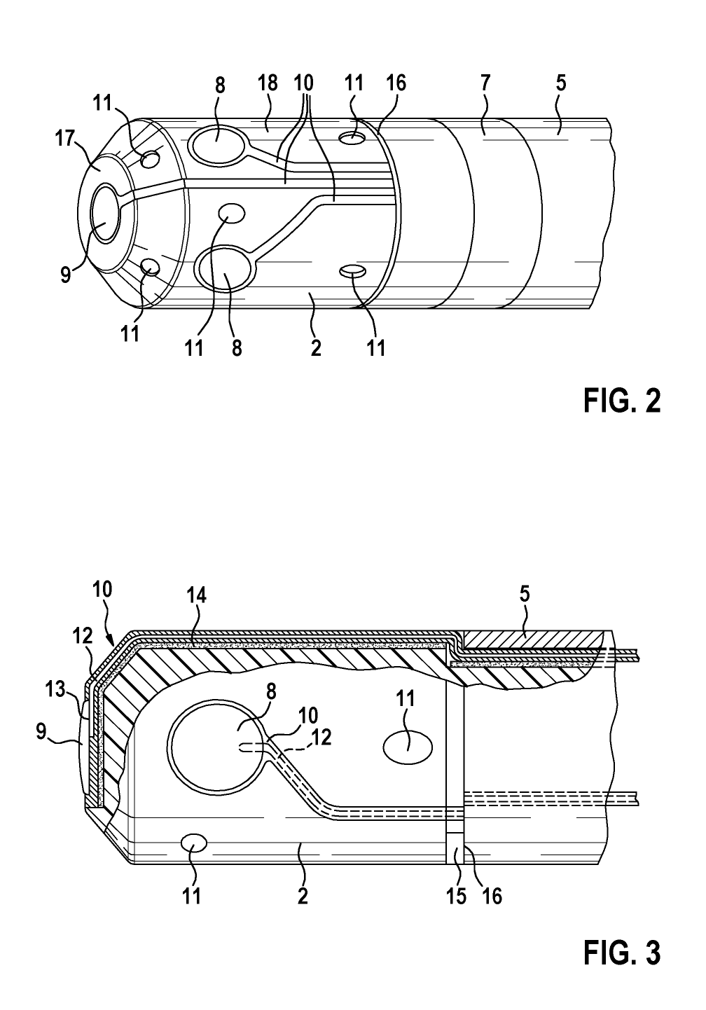

FIG. 1 schematically represents the ablation catheter 1 with an ablation electrode 2. The ablation electrode 2 is arranged at a distal end of a catheter shaft 5. The ablation electrode 2 has lateral microelectrodes 8 arranged on a lateral surface 18 (FIG. 2) of the ablation electrode 2 and a frontal microelectrode 9 arranged on an end face 17 (FIG. 2) of the ablation electrode 2. There can also be multiple frontal microelectrodes (not shown) arranged on the end face 17. Proximal of the ablation electrode 2 there are three ring electrodes 7 for the conventional mapping process. At the proximal end of the catheter shaft 5 there is a catheter handle 4. The ablation electrode 2, the lateral microelectrodes 8, the frontal microelectrode 9, and the ring electrodes 7 are connected, via the catheter shaft 5 through the catheter handle 4, with terminals in a plug 3. The plug 3 provides a connection for the ablation electrode 2, the lateral microelectrodes 8, the frontal microelectrode 9, and the ring electrodes 7, and possibly a thermocouple to a controller. An irrigation connection 6 for irrigating the ablation electrode 2 is brought out from the catheter handle 4.

FIG. 2 shows the catheter head with the ablation electrode 2 and a part of the catheter shaft 5 and one of the ring electrodes 7. The ablation electrode 2 is equipped with a frontal microelectrode 9 and four lateral microelectrodes 8. The microelectrodes 8, 9 are electrically insulated from the ablation electrode 2 by LCP films 10. Each LCP film surrounds a conductor track, every microelectrode 8, 9 being connected with a conductor track (not shown). The LCP films 10 are arranged in recesses in the ablation electrode 2. The depth of the recesses is 0.05 mm. The microelectrodes 8, 9 and the LCP films 10 are flush with the surface of the ablation electrode 2. The LCP films 10 are routed on the surface of the ablation catheter all the way to the proximal end of the ablation catheter 2. At the proximal end, the LCP films (with the conductor tracks) are routed into an interior of the catheter shaft 5. The LCP films 10 can be folded or bent like paper, and thus can easily be guided around an edge of the ablation electrode 2 by gluing 15 in the catheter shaft 5. The LCP films 10 are shaped so as to bypass the irrigation openings 11 by a good distance, and thus keep them open.

FIG. 3 shows the ablation electrode 2 from FIG. 2 cut in such a way that the cross section of a recess is visible all the way to the frontal microelectrode 9. Here the LCP film 10 is connected with the ablation electrode 2 by means of an adhesive 14 for the microelectrode 9 at the bottom of the recess. The LCP film 10 contains a conductor track 12 (e.g., a copper conductor track), whose distal end has an electrically conductive connection with an exposed gold-plated electrode 13 (e.g., a copper electrode). The conductor track 12 is drawn with dashed lines, since it is completely surrounded by the LCP film 10 (and therefore is not directly visible). In the proximal direction, the conductor track 12 is routed through and electrically insulated from the catheter shaft 5 and the catheter handle 4, and is finally connected with a corresponding terminal of the plug 3.

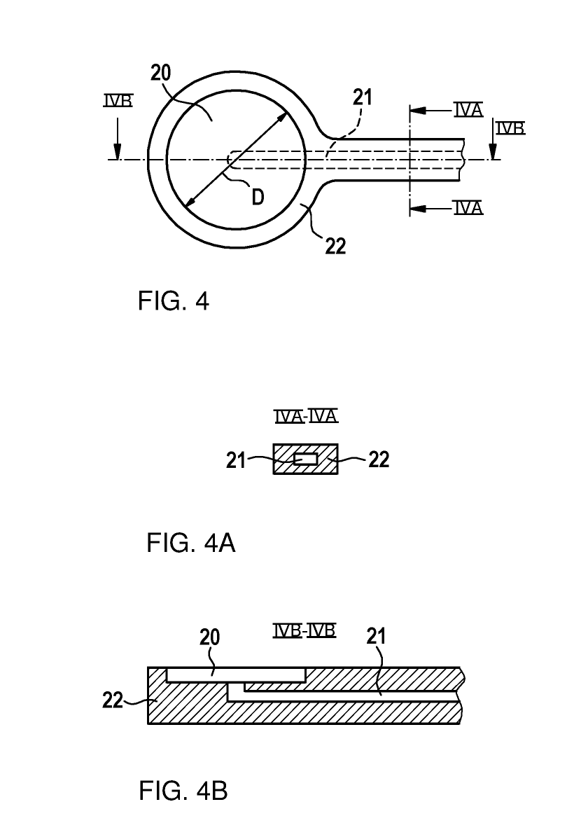

FIG. 4 shows a schematic representation of a microelectrode 20. In the embodiment shown, the microelectrode 20 has a circular surface with a diameter D (e.g., D=0.3 mm). The microelectrode 20 is electrically insulated from the ablation electrode (not shown) by an insulating material 22. The insulating material 22 can be, for example, an LCP film. A lead element 21 (e.g., a copper lead) is surrounded by the insulating material, as is shown for the cross section along the line IVA-IVA. In addition, a section is shown along the line IVB-IVB. The bottom of the microelectrode 20 is connected with the lead element 21.

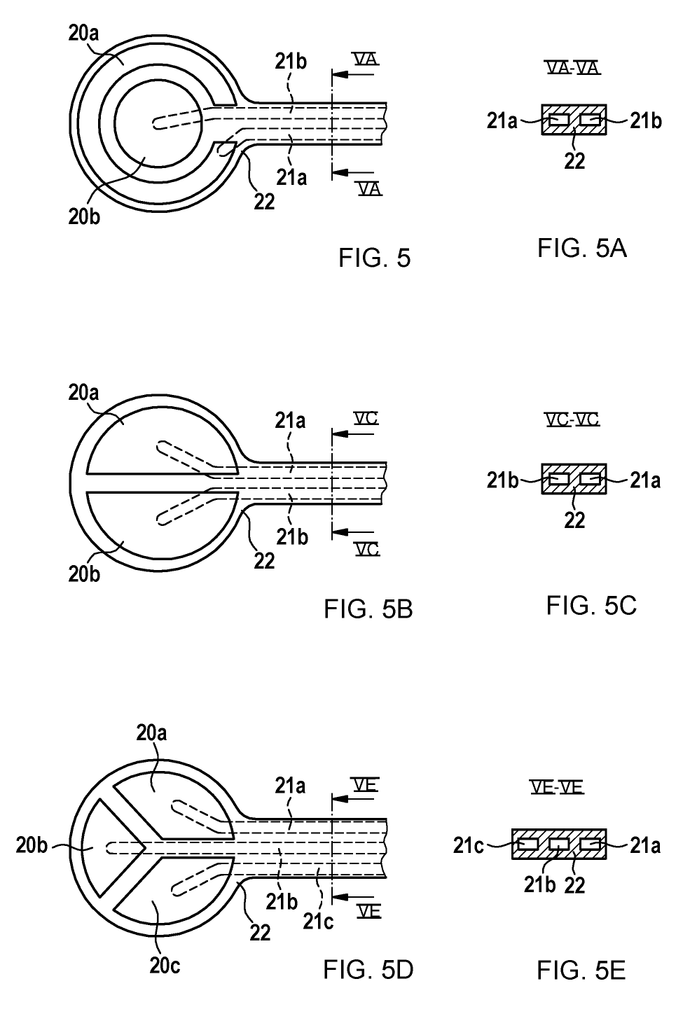

FIGS. 5, 5B and 5D show schematic representations of embodiments of a multipart microelectrode with corresponding sectional views, FIGS. 5A, 5C and 5E

FIGS. 5 and 5A shows a concentric microelectrode with two segments 20a, 20b. A first segment 20a forms an open or a closed circle that surrounds a second segment 20b in the form of a circular area. The two segments 20a, 20b are connected with lead elements 21a, 21b. The two segments 20a, 20b and the lead elements 21b, 21b are insulated from the ablation electrode (not shown) by the insulating material 22.

FIGS. 5B and 5C show a two-part microelectrode with two semicircular segments 20a, 20b. The two segments 20a, 20b are connected with lead elements 21a, 21b. The two segments 20a, 20b and the lead elements 21b, 21b are insulated from the ablation electrode (not shown) by the insulating material 22.

FIGS. 5D and 5E show a microelectrode with three segments 20a, 20b, 20c. The three segments 20a, 20b, 20c are connected with lead elements 21a, 21b, 21c. The three segments 20a, 20b, 20c and the lead elements 21a, 21b, 21c are insulated from the ablation electrode (not shown) by the insulating material 22.

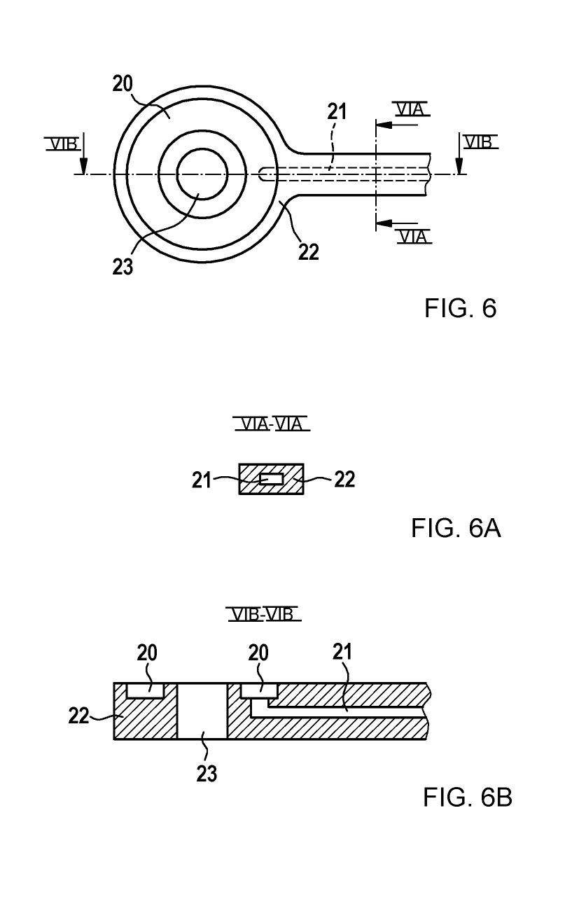

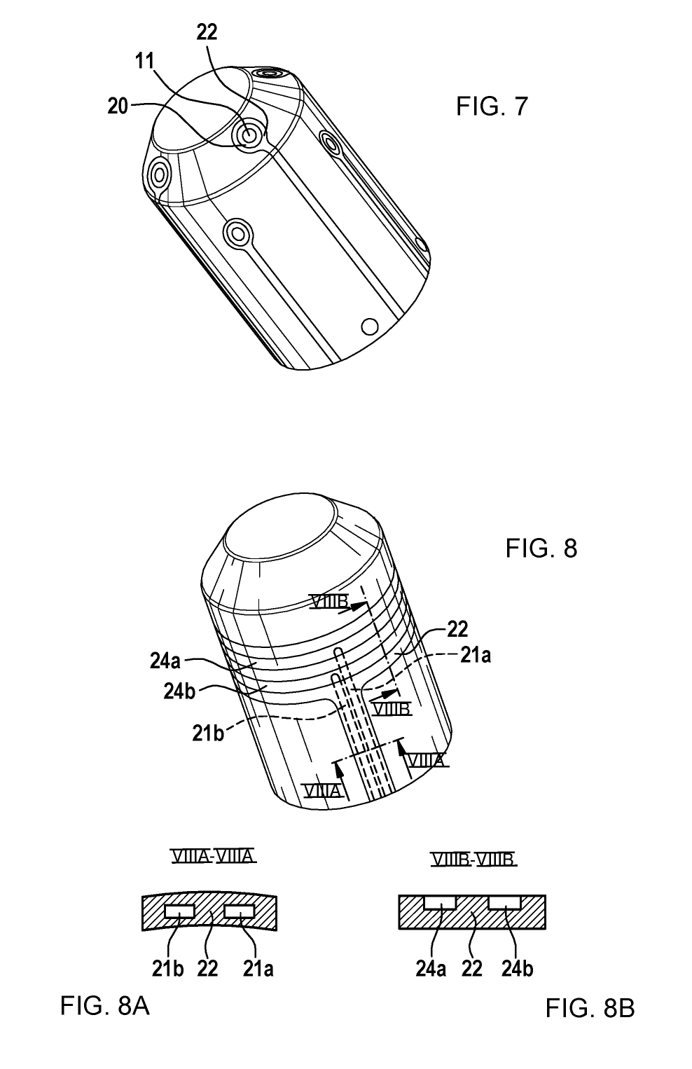

FIGS. 6, 6A and 6B show a microelectrode 20 with a hole 23. The microelectrode 20 is connected with the lead element 21. The microelectrode 20 and the lead element 21 are insulated from the ablation electrode (not shown) by the insulating material 22. In addition, sections views taken along the lines VIA-VIA and VIB-VIB are shown. FIG. 7 shows a catheter head of an irrigated catheter with microelectrodes 20 according to FIG. 6. The irrigation opening 11 leads through the hole in the microelectrode 20.

FIG. 8 shows a catheter head with ring microelectrodes 24a, 24b. The two ring microelectrodes 24a, 24b are connected with lead elements 21a, 21b. The two ring microelectrodes 24a, 24b and the lead elements 21a, 21b are insulated from the ablation electrode by the insulating material 22. As is shown in the section view of FIG. 8A, the lead elements 21a, 21b are completely surrounded by the insulating material. The sectional view of FIG. 8B shows the ring microelectrodes 24a, 24b embedded in the insulating material 22.

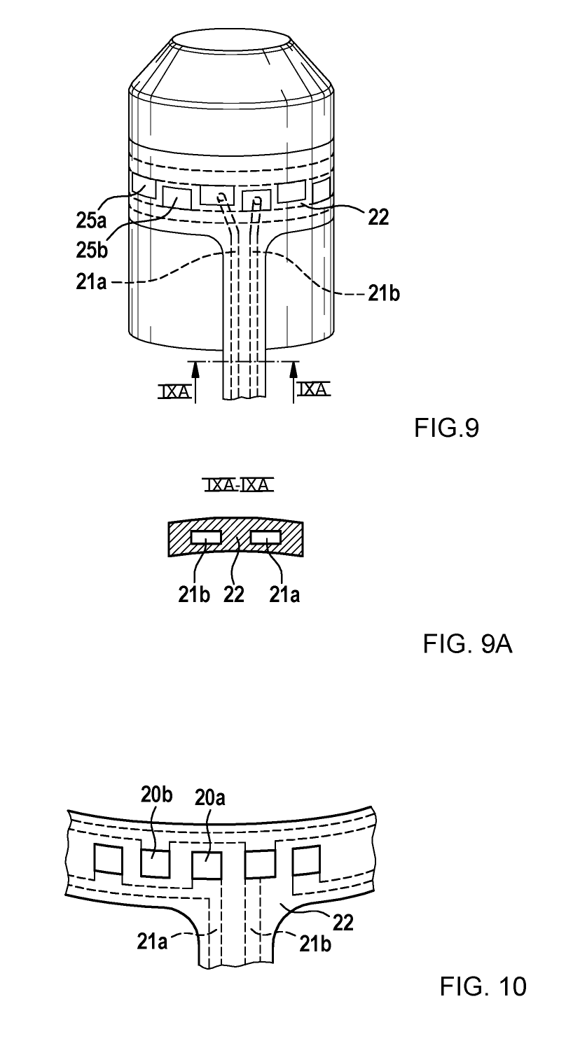

FIG. 9 shows a catheter head with toothed microelectrodes 25a, 25b. The two toothed microelectrodes 25a, 25b are connected with lead elements 21a, 21b. The two toothed microelectrodes 25a, 25b and the lead elements 21b, 21b are insulated from the ablation electrode by the insulating material 22. As is shown in the sectional view of FIG. 9A, the lead elements 21a, 21b are completely surrounded by the insulating material. FIG. 10 shows the connections of the toothed microelectrodes 25a, 25b to the lead elements 21a, 21b. The two lead elements 21a, 21b are connected with connection elements. This allows the toothed microelectrodes to be connected with only two lead elements 21a, 21b. Other tooth shapes are possible, such as, for example, triangular teeth or semicircular teeth.

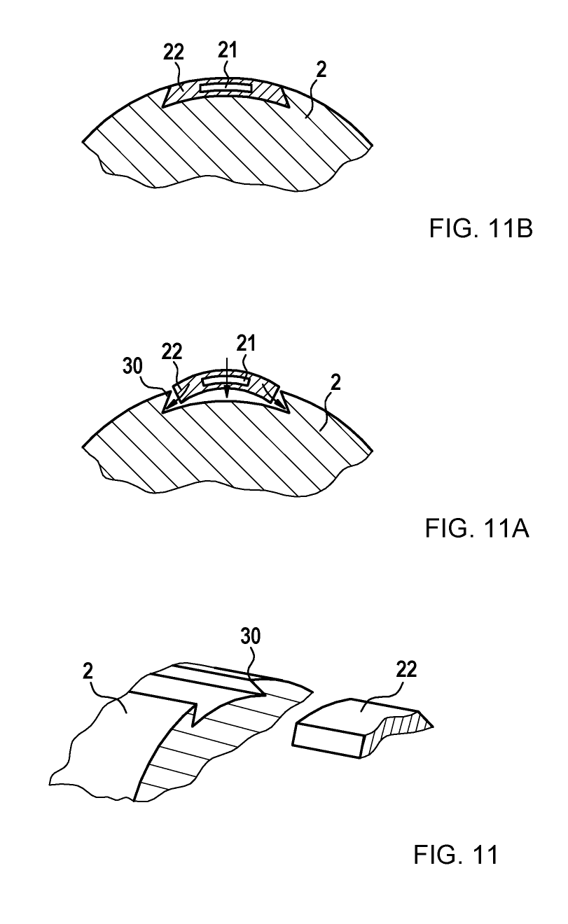

FIGS. 11-11B show a way of fastening the insulating material 22 without gluing. Instead, the insulating material 22 hooks in behind an edge 30 (undercut clamping). The insulating material (and/or the microelectrode) can be clipped into a recess, so that it catches behind the edge 30 (FIG. 11A), or it can be pushed into the recess (bottom picture of FIG. 11).

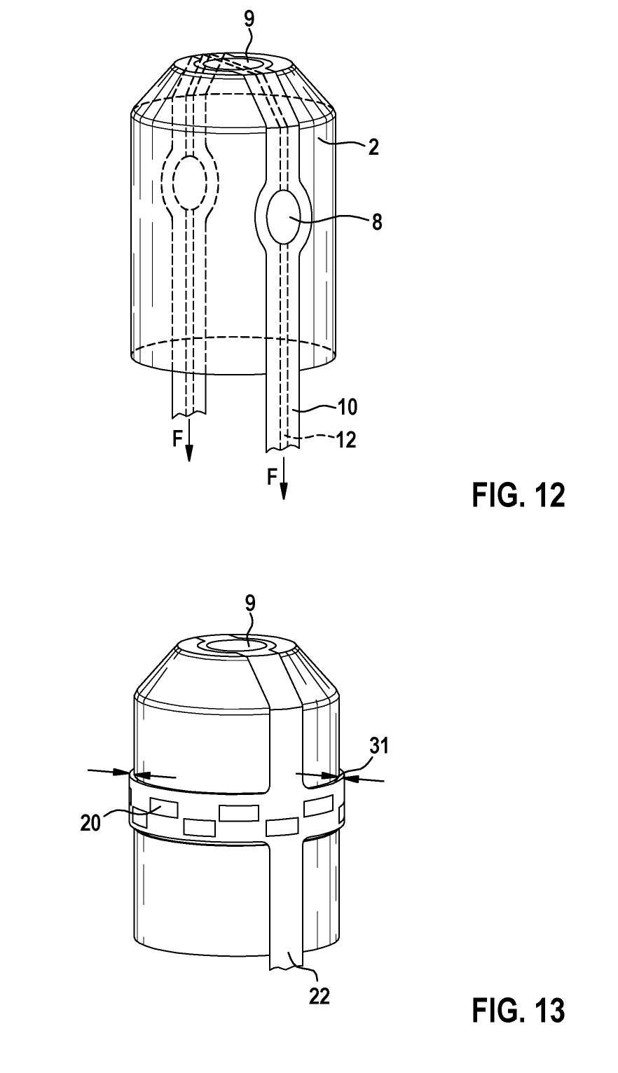

FIG. 12 shows a way of fastening by means of tensioning in a groove. The LCP film 10 with the conductor track 12 is tensioned around the catheter head on the ablation electrode 2. This fixes the conductor track 12 and the microelectrodes 8, 9.

FIG. 13 shows a way of fastening by means of shrinking. This involves the ablation catheter briefly being cooler than the microelectrodes 20 with their lead 21. The microelectrodes with the leads and the insulating material are arranged in a groove 31. After cooling, the microelectrodes sit tightly on the ablation electrode. Alternatively, it is also possible for the microelectrodes with the insulating material and the leads to be expanded and then engage in the groove (not shown).

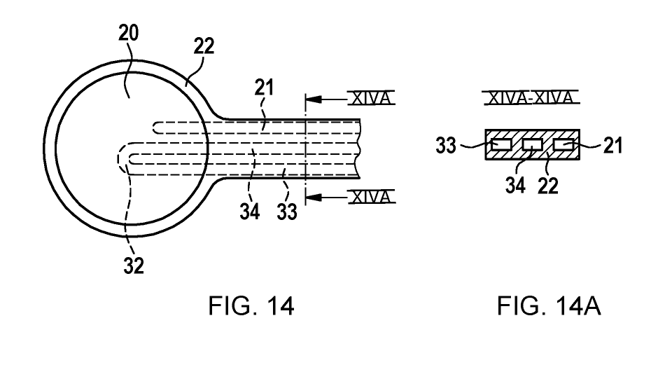

FIGS. 14 and 14A show a microelectrode 20 combined with a thermocouple 32 and the leads connected with the thermocouple 32, a copper wire 33, and a CuNi wire 34 (constantan, a copper-nickel alloy). The copper wire 33 and the CuNi wire 34 are embedded into the insulating material 22.

The ablation catheter according to the disclosed embodiments can have the following advantages:

1. The microelectrodes require very little space on the ablation electrode, especially for the fastening, the electrical insulation, and for making contact with the electrical leads.
2. The shape and mounting of the electrical lead routes can be managed relatively simply by gluing, folding, bending, and especially advantageously by tensioning, and it does not interfere with the routing of the irrigation lines and irrigation channels in the ablation electrode.
3. Using multiple microelectrodes can ensure that at least one microelectrode is always in contact with the tissue during the ablation.
4. Especially the embodiment of an ablation catheter with partial LCP surfaces is biocompatible and EO sterilizable.
5. An electrode with a hole allows better use of the surface area and other placement possibilities for microelectrodes and protection from overheating and the resulting formation of thrombi at the material transitions from the insulation edge (polymer) to the microelectrode and ablation electrode.

The features disclosed in the description, the claims, and the figures can be relevant, both individually and in any combination with one another, for the realization of embodiments.

The following is a summary list of reference numerals and the corresponding structure used in the above description of the invention:

1 Ablation catheter
2 Ablation electrode

### 3 Plug

4 Catheter handle
5 Catheter shaft
6 Irrigation connection
7 Ring electrodes
8 lateral Microelectrode
9 frontal Microelectrode
10 LCP film
11 Irrigation opening
12 Conductor track

### 13 Electrode

14 Adhesive for the microelectrode
15 gluing of the ablation electrode with the catheter shaft
16 Distal end of the catheter shaft
17 End face of the ablation electrode
18 Lateral surface of the ablation electrode
20(a,b,c) Microelectrode
21(a,b,c) Lead element
22(a,b,c) Insulating material

### 23 Hole

24(a,b) Ring microelectrode
25(a,b) Toothed microelectrode

### 30 Edge

### 31 Groove

### 32 Thermocouple

33 Copper wire
34 CuNi wire

1. An ablation catheter, comprising
a catheter shaft;
an ablation electrode disposed at a distal end of said catheter shaft;
a microelectrode disposed on a surface of said ablation electrode;
a lead element having an electrically conductive connection with said microelectrode; and
an insulating material surrounding said lead element so that said lead element is electrically insulated from said ablation electrode, at least sections of said insulating material with said lead element being disposed on said surface of said ablation electrode, and said lead element being fastened by means of tensioning.

2. The ablation catheter according to claim 1, wherein:
said surface of said ablation electrode having a recess formed therein; and
at least sections of said lead element surrounded by said insulating material are disposed in said recess formed on said surface of said ablation electrode.

3. The ablation catheter according to claim 1, wherein:
said surface of said ablation electrode having a recess formed therein; and
said microelectrode and said lead element surrounded by said insulating material are disposed in said recess formed on said surface of said ablation electrode.

4. The ablation catheter according to claim 2, wherein at least one of said microelectrode or said lead element surrounded by said insulating material is glued in said recess.

5. The ablation catheter according to claim 1, wherein said microelectrode is electrically insulated from said ablation electrode by said insulating material.

6. The ablation catheter according to claim 1, wherein said insulating material is a flexible film material.

7. The ablation catheter according to claim 1, wherein said insulating material is a liquid crystal polymer.

8. The ablation catheter according to claim 1, wherein at least one of said microelectrode or said lead element surrounded by said insulating material are flush with said surface of said ablation electrode.

9. The ablation catheter according to claim 1 wherein said lead element surrounded by said insulating material is disposed on said surface of said ablation electrode from said microelectrode all the way to said distal end of said catheter shaft, and is routed in an interior of said catheter shaft at said distal end of said catheter shaft.

10. The ablation catheter according to claim 1,
further comprising an irrigation tube disposed in an interior of said catheter shaft; and
wherein said ablation electrode has an irrigation opening formed therein, and said irrigation opening is connected with said irrigation tube.

11. The ablation catheter according to claim 10, wherein said microelectrode has a hole formed therein, and is disposed on said surface of said ablation electrode in such a way that at least sections of said hole of said microelectrode are disposed on said irrigation opening.

12. The ablation catheter according to claim 1, wherein said microelectrode is a ring electrode that surrounds said ablation electrode around its periphery.

13. The ablation catheter according to claim 1, wherein said microelectrode is a toothed microelectrode.

14. A method for producing an ablation catheter, which comprises the steps of:
providing a catheter shaft;
disposing an ablation electrode on a distal end of the catheter shaft;
disposing a microelectrode on a surface of the ablation electrode; and
providing a lead element that has an electrically conductive connection with the microelectrode, the lead element being surrounded by an insulating material so that the lead element is electrically insulated from the ablation electrode, wherein at least sections of the insulating material with the lead element are disposed on the surface of the ablation electrode, and wherein the lead element is fastened by means of tensioning.
